(19) United States
(12) Patent Application Publication
(10) Pub. No.: US 2007/0259056 A1
(43) Pub. Date: Nov. 8, 2007

(54) Anti-Tumour Terpene Compounds

(75) Inventors: Ifor Delme Bowen, Cardiff (GB); Ahmed Yasine Ali, Cardiff (GB)

Correspondence Address: BANNER & WITCOFF, LTD., 1100 13th STREET, N.W., SUITE 1200, WASHINGTON, DC, 20005-4051, US

(73) Assignee: COMPTON DEVELOPMENTS LTD., Swansea (GB)

(21) Appl. No.: 10/579,135

(22) PCT Filed: Nov. 12, 2004
(86) PCT No.: PCT/GB04/04769

§ 371 Date: Apr. 11, 2007

(30) Foreign Application Priority Data
Nov. 13, 2003 (GB) 0326518.8

(51) Int. Cl. A61K 31/01; A61K 31/015; A61K 36/328; A61P 35/00; A61P 35/02

(52) U.S. Cl. 424/748; 514/762; 514/763

(57) ABSTRACT

The present invention relates to the use of a terpene compound in the treatment or prophylaxis of cancer. The present invention also relates to a pharmaceutical composition comprising the terpene compound and a pharmaceutically acceptable excipient, carrier, adjuvant or vehicle.

The present invention relates to the use of a terpene compound in the treatment or prophylaxis of cancer. The present invention also relates to a pharmaceutical composition comprising the terpene compound and a pharmaceutically acceptable excipient, carrier, adjuvant or vehicle.

Myrrh, the resinous product of the African shrub, Commiphora molmol, has a long folk history of healing. The medicinal background of Myrrh and scented Myrrh (Commiphora guidotti) is largely based on ancient folklore and has found general use in Arab, Indian and Chinese herbal remedies. In this context it is known to be non-toxic and to generally exhibit anti-inflammatory, antiseptic and healing properties. It is also widely used in the perfume and flavouring industries. Its specific anti-tumour properties have not been systematically or scientifically tested, although there has been a preliminary report of the activity of the crude extract of Commiphora molmol against Ehrlich solid tumour in mice (Al Harbi et al., Experimental Chemotherapy, 40, 337-347, 1994). The crude extract is also cited in a number of herbal remedies for use in immunological disorders and against Schistosomiasis.

The activity of individual chemical components of C. molmol and C. gudiotti has not been analysed. Our initial chemical analysis of extracts shows them to be a very rich source, comprising of some 300 compounds.

In terms of background information, the use of Myrrh in many herbal remedies is known but no prior art documents specify the systematic identification of specific anticancer chemical compounds from Myrrh. A few references indicate the efficacy of a crude extract of Myrrh (Commiphora molmol) against a mouse tumour (Qureshi & Al Harbi et al., Cancer Chemotherapy and Pharmacology, 33, 130-138, 1993; and Al-Harbi et al., 1994 (supra)).

International Patent Application WO 00/50053 describes the use of Myrrh (Commiphora molmol) with other plants as a classical herbal treatment of “cancer”, again using crude extracts. A further publication (Rao et al., J. Ethnopharmacology, 76, 151-154, 2001) describes the non toxic impact of Commiphora molmol on mice.

Tatman et al., (Cancer Leffers, 175, 129-139, 2002) discloses that volatile isoprenoids are active against murine melanoma and human leukemia cell lines.

Leal et al., (J. Agric. Food. Chem., 51, 2520-2525, 2003) discloses that ginger and tumeric extracts showed anti-cancer activities.

Saleh et al., (Pharm. Pharmacol. Lett., 8, 97-99, 1998) discloses that volatiles of celery are cytotoxic against human cancer cell lines.

US patent U.S. Pat. No. 6,486,706 discloses the use of α- and β-santatols in the treatment of viral-induced tumours.

US patent U.S. Pat. No. 5,602,184 discloses the use of selected terpenes against human cancer cell lines.

US patent application US 2002/0155522 discloses the use of β-elemene against tumour cells.

International PCT application WO 01/80868 disclose the use of sesquiterpene mixtures for preventing and treating cancers.

Itokawa et al., (Chem. Pharm. Bull., 33, 3488-3492, 1985) describes studies on the anti-tumour activity of bisabolene sesquiterpenoids.

A range of compounds obtained from C. molmol and/or C. gudiotti can be gauged from the spectra or peaks of chemicals obtained by GC/MS and HPLC/MS. These peaks can be sampled giving a wide range of new molecules to be tested. Some of the innovative aspects of the work include: 1) choosing the biomedically rich source of untested molecules in Myrrh (Commiphora molmol) and Haddi or scented Myrrh (Commiphora guidotti), 2) the identification and characterisation of a wide source of novel compounds from these Myrrh extracts, and 3) the use of specific (apoptotic) bioassays to select non-toxic compounds suitable for controlling human tumour cell lines.

None of the prior art documents disclose any specific compound from Myrrh or scented Myrrh having anti-cancer activity against human tumours. As will be appreciated, there is demand for effective anti-cancer drugs and in particular for drugs that have no, or limited, toxicity against non-cancerous cells. The inventors have identified a number of specific compounds that specifically induce apoptosis in human tumour cells but not in normal tissues.

Current treatments using drugs such as Taxol, Paclitaxel and Vinblastin result in a damaging form of inflammatory cell death or necrosis. Drugs selected for apoptosis, which is a natural process of programmed cell death, should induce little or no side effects. This approach is innovative in that it selects for a benign mechanism of action that should be attractive to both the pharmaceutical industry and ultimately to the medical profession. This selective approach has revealed non-toxic anticancer agents. According to a first aspect of the present invention there is provided the use of a compound in the manufacture of a medicament for the treatment or prophylaxis of cancer, wherein the compound is a terpene or oxygenated derivative thereof, and the terpene is selected from:

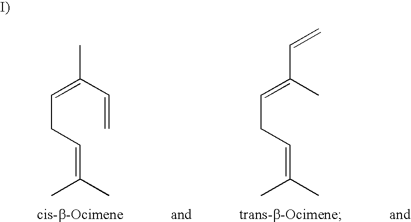

II) Monocyclic sesquiterpenes of the bisabolene type.

According to a second aspect of the present invention, there is provided a method of treating or preventing cancer comprising administering to a patient in need of such treatment an effective dose of a compound, wherein the compound is a terpene or oxygenated derivative thereof, and the terpene is selected from:

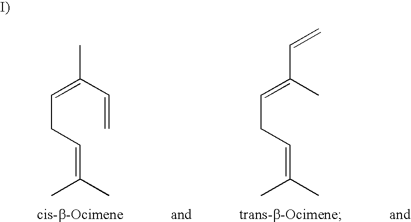

II) Monocyclic sesquiterpenes of the bisabolene type.

It has been found that the specific terpene compounds referred to above act as anti-cancer agents by inducing apoptosis of human cancer cells.

Preferably the compound is trans-β-ocimene.

Representative terpenes of the bisabolene type include:

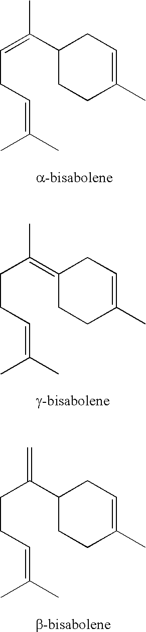

Preferably the terpene of the bisabolene type is α-bisabolene or γ-bisabolene.

In one preferred embodiment of the present invention the terpene is not α-bisabolol.

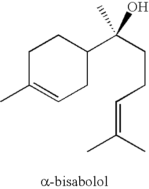

It is particularly preferred that the compound used in the first or second aspect of the present invention is selected from the representative compounds indicated above. Minor modifications within the general formulae of the terpenes of the present invention can be made provided the modified compound has anti-cancer activity. Minor modifications including replacing a side group with a closely related side group that would not be considered to destroy the activity of the compound, e.g. replacing —CH₃ with —CH₂CH₃. Those skilled in the art are well aware of appropriate modifications that can be made to the terpene compounds encompassed by the general formulae given above without destroying the activity of the compounds.

It is particularly preferred that the terpene compound is trans-β-ocimene, -γ-bisabolene or α-bisabolene.

The compound used in the first or second aspect the present invention can be obtained from a plant in the genera Detarium, Ximenia, Polygonum, Commiphora or Boswellia. In particular, it is preferred that the compound can be obtained from the plant Detarium microcarpum, Ximenia americana, Polygonum limbatum, Commiphora molmol, Commiphora guidotti or a Boswellia sp.

The compound used in the first or second aspect of the present invention can be obtained by isolating it from the appropriate plant indicated above. In particular, the majority of the compounds can be isolated from Commiphora molmol or Commiphora guidotti. The compound can be isolated using any suitable preparative method. Methods of isolating such compounds are known to those skilled in the art and include chromatographic methods such as flash column chromatography and solid phase extraction columns. Nuclear Magnetic Resonance and Mass Spectrometry can be used to identify the individual compounds.

When the compound used in the composition of the present invention is isolated from a plant, substantially no contaminating plant material is present. Substantially no contaminating plant material means that less than 0.1% (w/w) of contaminating plant material is present.

The compound used in the composition of the present invention may be obtained from commercial sources such as RC Treat Ltd (trans-β-Ocimene and γ-bisabolene) and KIC Inc.; (α-bisabolol). The compound of the present invention may also be synthesised using standard chemical synthesis procedures.

Two or more of the terpene compounds may be used together as an anti-cancer agent. Furthermore, additional anti-cancer agents, therapeutics, markers, etc. can also be used in conjunction with the terpene compounds used according to the first or second aspect of the present invention.

The term “anti-cancer” agent refers to an agent that can be used to prevent or reduce the development of a cancer or that can reduce the size of a cancer.

The cancer to be prevented or treated according to the first and/or second aspect of the present invention can be any cancer, but is preferably a human cancer. Preferred cancers include breast cancer, ovarian cancer, prostate cancer, colon cancer, lung cancer, pancreatic cancer, bowel cancer, melanoma, testicular cancer, cervical cancer, fibrosarcoma, squamous cell carcinoma, leukemia, astrocytoma and glioma.

It has been found that the compound used in the first and second aspects of the present invention produces a differentially higher level of apoptosis in cancer cells than in corresponding normal cells. This means that at suitable doses the compounds are differentially active against cancer cells.

According to a third aspect of the present invention there is provided a pharmaceutical composition comprising a terpene compound or oxygenated derivative thereof, wherein the terpene is selected from:

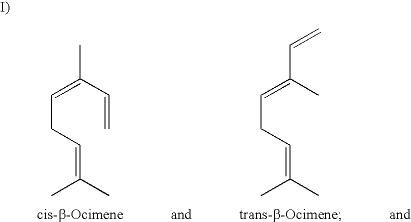

II) monocyclic sesquiterpenes of the bisabolene type, in combination with a pharmaceutically acceptable excipient, carrier, adjuvant or vehicle.

Preferably the terpene compound is as defined above with respect to the first and second aspect of the present invention.

The terpene compound can be delivered to an individual in combination with any pharmaceutically acceptable carrier, adjuvant or vehicle. Pharmaceutically acceptable carriers, adjuvants and vehicles that may be used include, but are not limited to, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protomine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene- polyoxypropylene- block polymers and wool fat.

The pharmaceutical composition may be administered orally, parentally, by inhalation spray, topically, rectally, nasally, buccally, vaginally or by an implanted reservoir. Preferably, the pharmaceutical composition is administered by injection. The term “parenteral” as used herein includes subcutaneous, intracutaneous, intravenous, intramuscular, intra-articular, intrasynovial, intrasternal, intrathecal, intralesional and intracranial injection or inflision techniques.

The pharmaceutical composition may be delivered in the form of a sterile injectable preparation, for example as a sterile injectable aqueous or oleaginous suspension. This suspension may be formulated accordingio techniques known in the art using suitable dispersing or wetting agents (such as, for example, Tween 80) and suspending agents. The sterile injectable preparation may also be a sterile iniectable solution or suspension in a non-toxic parentally-acceptable diluent or solvent, for example as a solution in 1, 3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or di glycerides. Fatty acids such as oleic acid and its glyceride derivatives are useful in the preparation of injectables, as are naturally pharmaceutically acceptable oils such as olive oil or caster oil, especially in their polyoxyethyated versions. These oil solutions or suspensions may also contain a long chain alcohol diluent or dispersant such as Ph. Helv or a similar alcohol.

The pharmaceutical composition of the present invention may also be administered as a fluid or in the form-of suppositories for rectal administration. The suppository can be prepared by terpene compound with a suitable non-irritating excipient which is solid at room temperature but liquid at the rectal temperature and therefore will melt in the rectum to release the terpene compound. Such materials include but are not limited to cocoa butter, beeswax and polyethylene glycols.

Topical administration of the composition may be desirable when the desired treatment involves areas or organs readily accessible for topical application. For application topically to the skin, the terpene compound should be formulated with carriers for topical administration, such as, but not limited to mineral oil, liquid petroleum, white petroleum, propylene glycol, polyoxyethylene, polyoxypropylene compounds, emulsifying wax and water. Alternatively, the terpene compound can be formulated with a suitable lotion or cream, or dissolved in a carrier. Suitable carriers include but are not limited to mineral oil, sorbitan monosterate, polysorbate 60, cetyl esters, wax, cetearyl alcohol, 2-octyldodecanol, benzyl alcohol and water. The terpene compound can be applied topically to the lower intestinal tract by a rectal suppository formulation or as a suitable enema formulation.

The pharmaceutical composition of the present invention may be administered by nasal aerosol or inhalation. Suitable compositions for such administration can be prepared according to techniques well kcnown to those sliilled in the art of pharmaceutical formulation and can be prepared as solutions in saline, employing benzyl alcohol or other preservatives, absorption promoters to enhance bio-availability, fluorocarbons, and/or other solublising other dispersing agents known in the art.

The precise amount of the terpene compound to be delivered to a patient will depend upon the severity of the cancer state, the general health, age, weight and gender of the subject, diet, time and frequency of administration, reaction sensitivities, and tolerance/response to therapy. A suitable amount can be determined by routine experimentation and is within the judgement of the clinician. Generally, and effective dose will be from 0.2 μM to 100 μM, more preferably 0.2 μM to 10 μM.

The pharmaceutical composition of the present invention may be administered in combination with other drugs, agents or markers.

The present invention also provides the terpene compounds used in the first aspect of the present invention for use in therapy.

The present invention also provides the use of the terpene compounds used in the first aspect of the present invention as anticancer agents in vitro.

The present invention also provides the use of a Commiphora guidotti extract in the manufacture of a medicament for the treatment or prophylaxis of cancer.

The present invention also provides a method of treating or preventing cancer comprising administering to a patient in need of such treatment an effective dose of a Commiphora guidotti extract. The Commiphora guidotti extract can be any extract and preferably it contains at least one of the compounds used in the first aspect of the present invention. Preferably the extract contains at least one of trans-β-ocimene, γ-bisabolene and (α-bisabolene.

The extract is preferably an alcoholic extract and may be obtained using standard procedures for obtaining alcoholic extracts of the plant. In particular, methods for obtaining such an alcoholic extract are well known to those sldlled in the art. Alternatively, it is preferred that the plant material is an alcoholic extract of an essential oil of the plant. Essential oils are the volatile, organic constituents of fragment plant matter. Essential oils are generally extracted from plant by two main methods, distillation (steam, water or dry distillation) and cold pressing. A plant extract containing mainly oils can also be prepared using solvents, carbon dioxide extraction or hydrofluoroallcanes. Tincture plant extracts can be made by macerating the plant materials and extracting using aqueous, ethanolic solvents (70%-90% ethanol in water) and left for a period of time, after which the solid debris is filtered.

The following examples, with reference to the figures, are offered by way of illustration and are not intended to limit the invention in any manner.

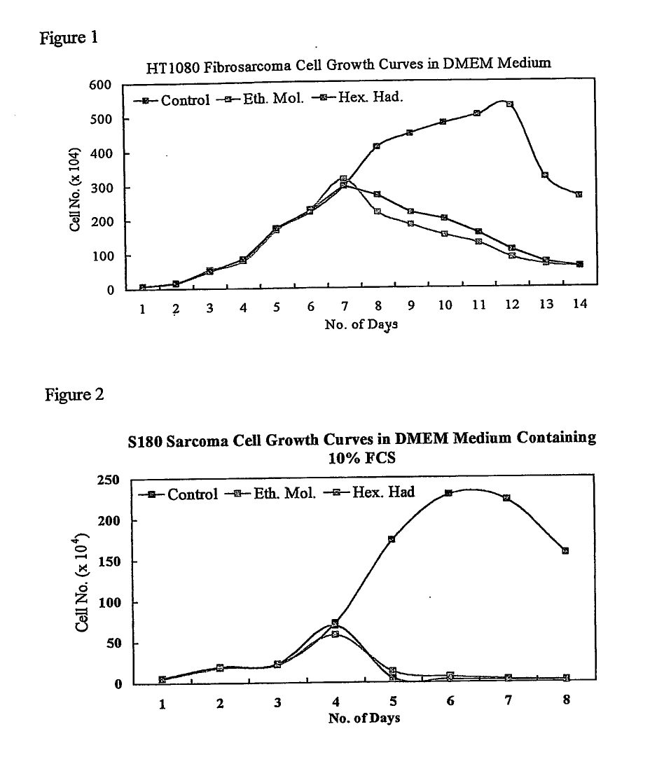

FIG. 1 shows the growth curve of human fibrosarcoma cells with and without addition of 667 ug/ml C. molmol and Haddi extracts to the growth medium.

FIG. 2 shows growth curve of S180 fibrosarcoma cells with and without the addition of 667 ug/ml C. molmol and Haddi extracts to the growth medium.

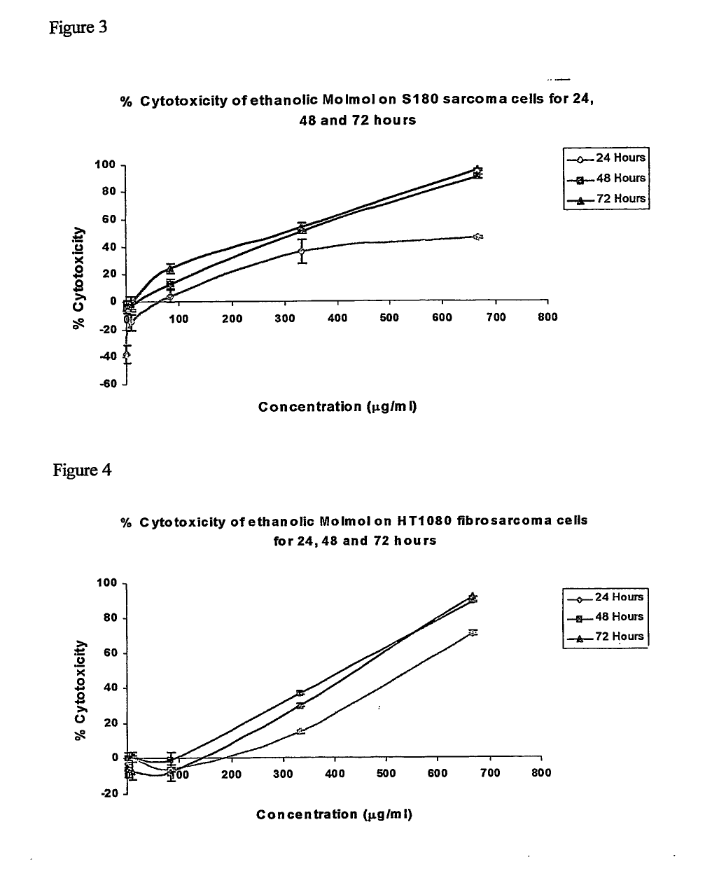

FIG. 3 shows the cytotoxic effects of ethanol extracted C. molmol on S180 fibrosarcoma cell lines.

FIG. 4 shows the cytotoxic effects of ethanol extracted C. molmol on HT1080 human fibrosarcoma cell lines.

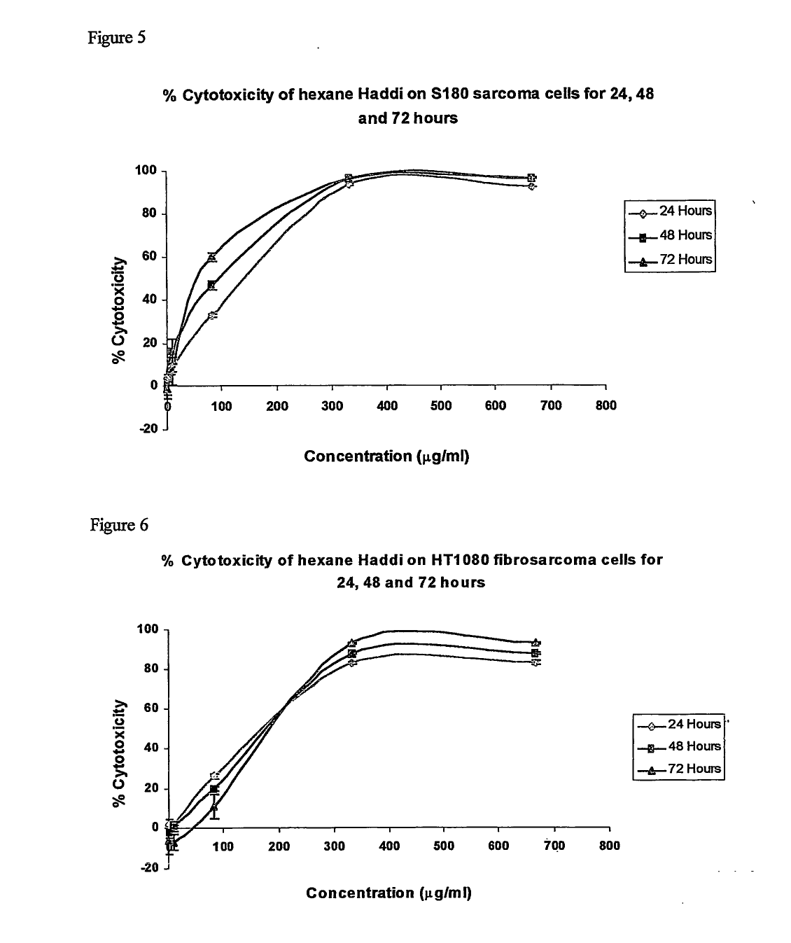

FIG. 5 shows the cytotoxic effects of hexane extracted Haddi on S180 fibrosarcoma cell lines.

FIG. 6 shows the cytotoxic effects of hexane extracted Haddi on HT1080 human fibrosarcoma cell lines.

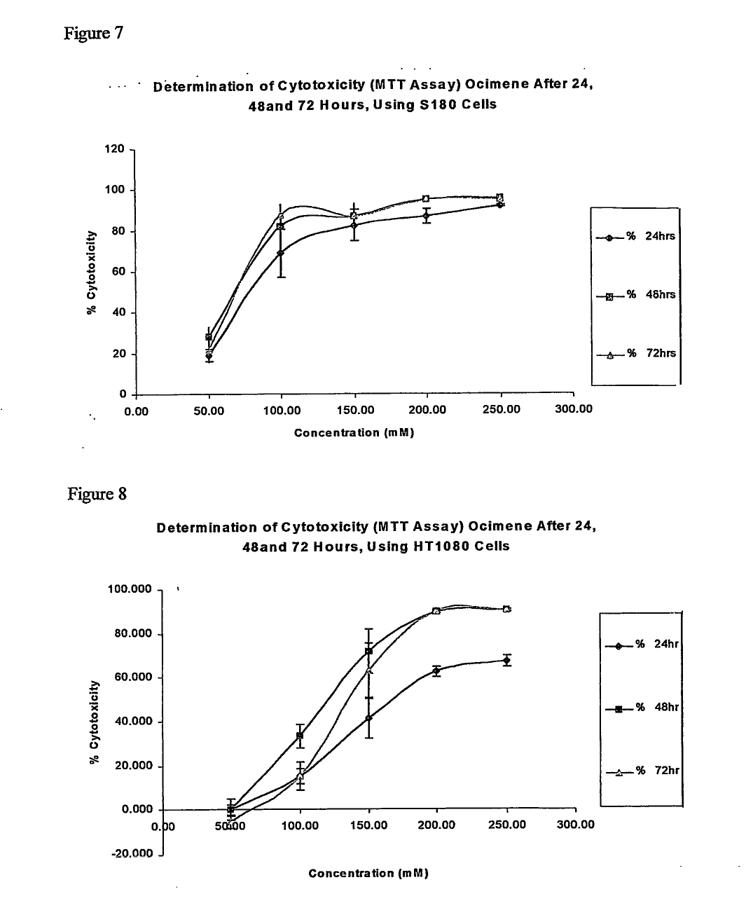

FIG. 7 shows the cytotoxic effects of trans-β-Ocimene S180 tumour cell lines.

FIG. 8 shows the cytotoxic effects of trans-β-Ocimene on HT1080 tumour cell lines.

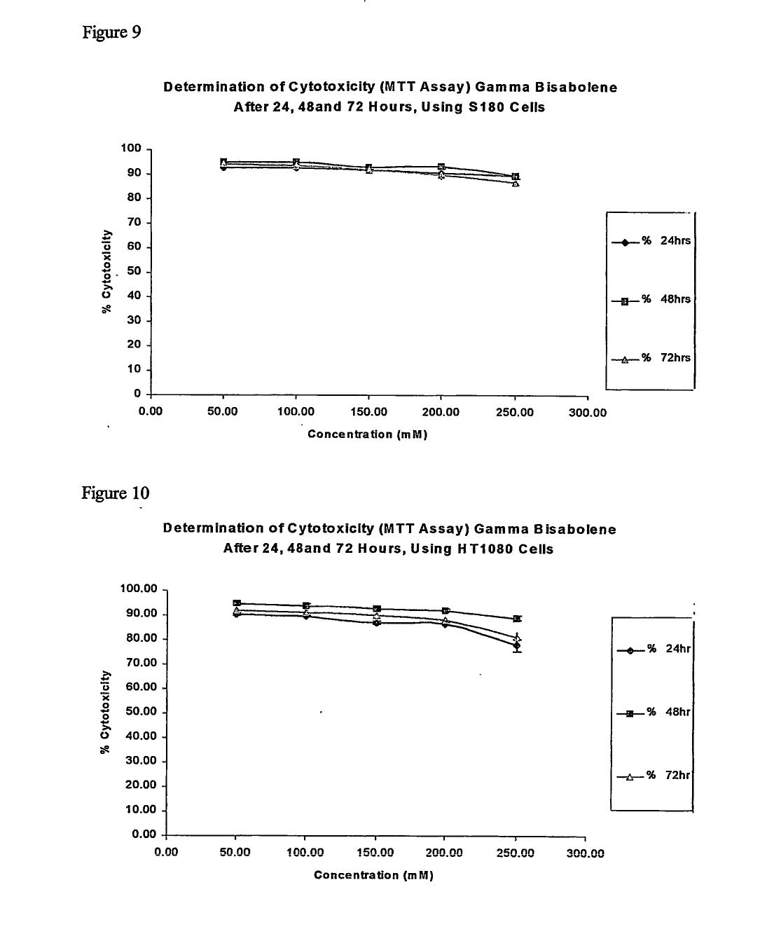

FIG. 9 shows the cytotoxic effects of γ-Bisabolene on S 180 tumour cell lines.

FIG. 10 shows the cytotoxic effects of γ-Bisabolene on HT1080 tumour cell lines.

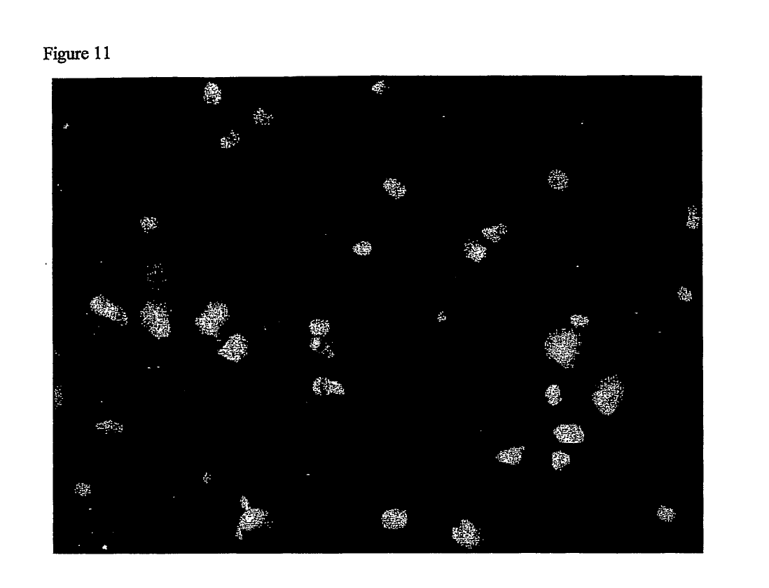

FIG. 11 shows the induction of apoptotic cell death in HT1080 human fibrosarcoma cells treated with 5 uM Bisabolene over 12 hours.

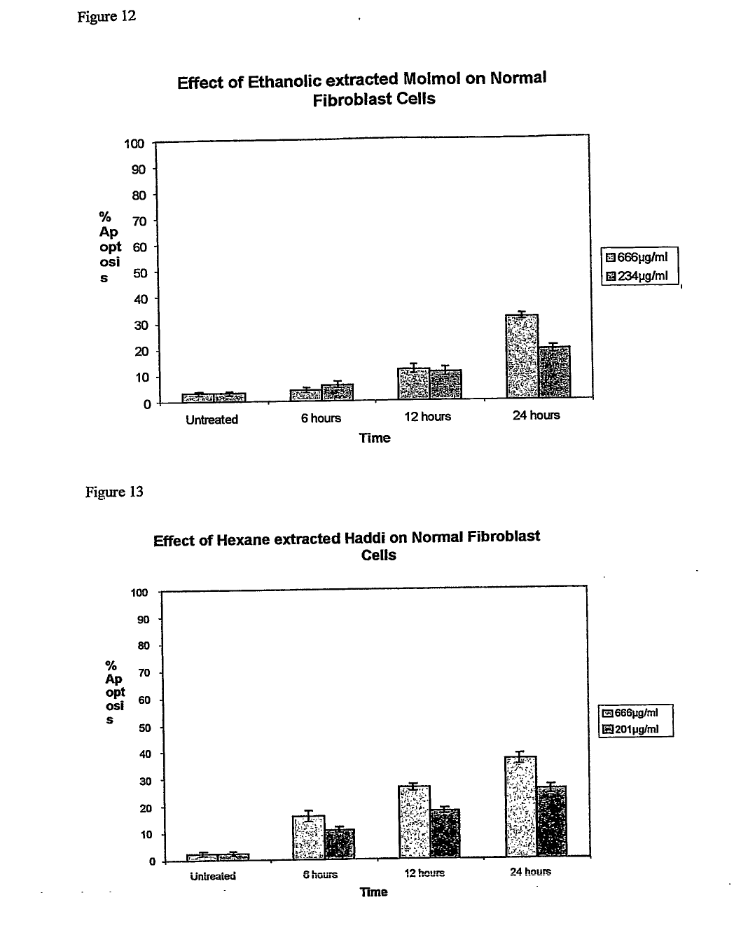

FIG. 12 shows the effect of ethanolic extracted C. molmol on normal fibroblast cells.

FIG. 13 shows the effect of hexane extracted Haddi on normal fibroblast cells.

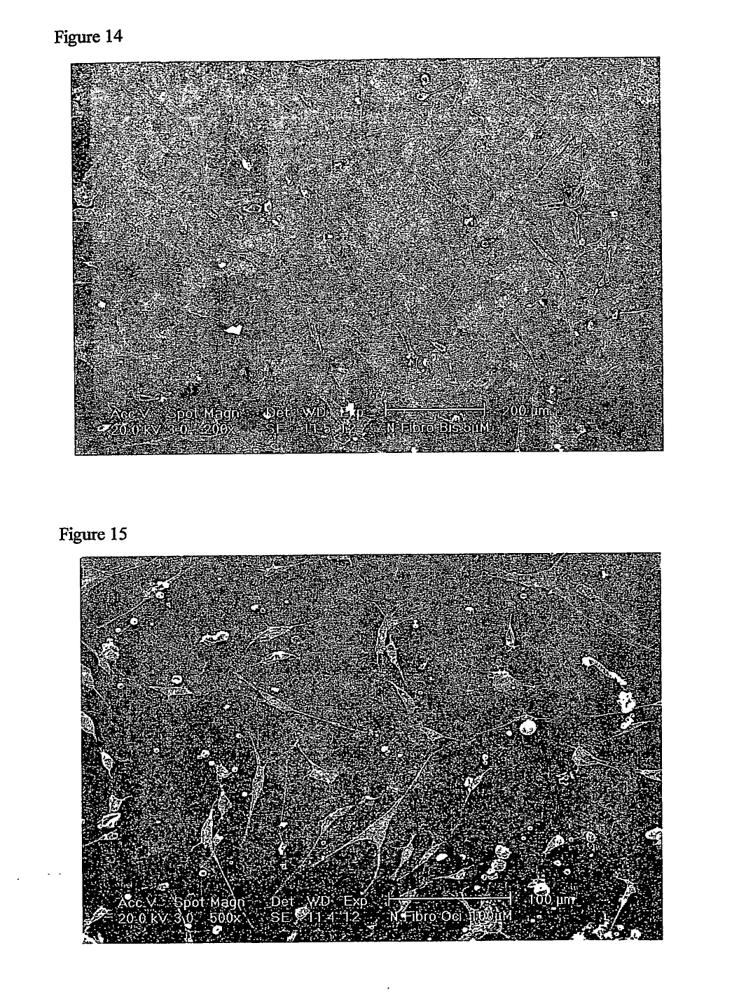

FIG. 14 shows a mixed tissue culture treated for 12 hrs with 5 uM Bisabolene.

FIG. 15 shows a mixed tissue culture treated for 12 hrs with 100 uM trans-β-Ocimene.

### EXAMPLES

1. Growth curve analyses provide vital information on a cell lines growth pattern over a period of time in vitro.

The cell death inducing properties of ethanolic extracted C. molmol and hexane extracted Haddi on the S180 murine sarcoma cell line and the human fibrosarcoma HT1080 cell line were tested. Results from a previous experiments had indicated that a 667 ug/ml solution of both extracts was potent as a cell-death-inducing agent. A stock cell suspension with a cell density of 5×10⁴ cells/ml was freshly produced from an existing cell culture. Forty-eight flasks were labeled and seeded with 5 ml of this cell suspension—2 control/untreated, 2 ethanolic C. molmol and 2 hexane Haddi flasks for each day of the 8 day experiment. All flasks were incubated at 37° C. and 5% CO₂. On day 4 the medium was changed in all remaining flasks. Untreated medium was used to replenish the control flasks while medium with an extract concentration of 667 ug/ml was used to replenish the relevant treatment flasks. Day 4 was chosen as the treatment day because this is the first day of the exponential growth phase of the growth curve. Treatment at this point in the growth curve would serve to fully demonstrate the ability of ethanolic C. molmol and hexane Haddi to induce cell death in this transformed cell line. Similar protocols were used to grow HT1080, a human fibrosarcoma cell line. In this instance growth was slower and cells were treated with extract when confluent at day 7 (See FIG. 1).).

Daily counts were carried out at the same time each day following standard protocol. Briefly, all medium was removed by pipetting. Flasks were then washed with 2 ml PBS twice to remove as much cell debris as possible from the flasks (this would aid in cell wash just to neutralise any remaining medium. All liquid was then carefully removed by pipetting before a further 1 ml of Tris-EDTA was added to the flasks. These flasks were incubated at 37° C. and 5% CO₂ for 2-5 minutes to ensure all cells had become detached from the flask. From day 4 onwards 2 ml of Tris-EDTA was used to ease cell counting as the cell number had increased significantly. This cell suspension was mixed by gentle pipetting to ensure homogeneity and a sample then placed in a hemocytometer and cells were counted under a light microscope. In all, 4 counts were taken from each flask. Mean values were calculated and used to plot the growth curves (FIGS. 1 and 2).

FIG. 2. clearly shows, S180 cells display a classical sigmoidal growth curve. Cell density increases very slowly between day 0 and day 3 from 5×10⁴ cells/ml to 21.1×10⁴ cells/ml. Day 3 to day 6 is the exponential growth phase when cell density increases 10-fold to 240×10⁴ cells/ml, with a doubling time of approximately 24 hrs during this phase. A plateau phase is observed between days 6 and 7 where cell number remains relatively constant and then begins to fall at day 8. This decrease in cell number is due to space and nutrient depletion in the cell culture.

Cells treated with hexane Haddi extract behave normally until day 4 (day of treatment). Day 5 sees a dramatic decrease in cell number to 1.8×10⁴ cells/ml from 68.6×10⁴ cells/ml on day 4. Cell number continues to decrease steadily reaching zero on day 8. This clearly demonstrates the ability of hexane extracted Haddi to induce cell death in rapidly growing transformed cell lines as the cells would normally be expected to continue growing until day 6 (see control curve).

Ethanolic C. molmol is also seen to be a potent inducer of cell death in S180 cells. Again, up to day 4 the S180 cells follow the typical growth pattern. Following treatment on day 4 the cell number falls from 47.9×10⁴ cells/ml (day 4) to 12.8×10⁴ cells/ml on day 5. The decrease in cell number occurs at a slower rate than with hexane Haddi treatment.

FIG. 1 shows similar response from human HT1080 fibrosarcoma cells following the addition of 667 ug/ml of ethanolic extracted C. molmol and hexane extracted Haddi to the growth medium on day 7. Untreated cells continue growing well up to day 13, whilst both C. molmol and Haddi treated cell lines decline rapidly in number from day 7.

2. MTT Assay for S180 sarcoma and HT1080 human fibrosarcoma cell lines treated with ethanolic C. molmol and hexane Haddi extracts

The MTT assay is a powerful, quantitative and highly reliable colourimetric assay that measures the viability of a cell population. The mitochondrial enzymes of viable cells are capable of metabolising 3-(4,5-diinethylthiazol-2-yl)-2,5-diphenyl tetrazolium bromide (MTT) from a yellow water-soluble compound to a water-insoluble purple formazan product—this is the chemical basis of the assay. The amount of formazan produced is directly proportional to the number of viable cells in the test population. The formazan product formed can be dissolved in dimethyl sulfoxide (DMSO), which enables quantification of the product to be obtained via spectrophotometry. A population containing largely viable cells will produce high levels of the formazan product and will therefore give a deep purple solution upon dissolution with DMSO, while a cell population that has mostly dead cells will produce a colourless solution. The amount of light absorbed (at a wavelength of 575 nm) can be converted to % cytotoxicity by using the following calculation:
[control absorption−test absorption]X 100=% cytotoxicity control absorption
Experimental Protocol

Two transformed cell lines were chosen for MTT analysis, S180 murine sarcoma cells and HT1080 human fibrosarcoma cells. Cells were seeded in 96-well microliter plates at a density of 2×10⁵ cells/ml and incubated at 37° C. and 5% CO₂ for 24 hours prior to treatment. 200 ul PBS was added to the wells on the outer edges of the plate to reduce evaporation loss from the wells containing cells.

In this study we tested ethanolic C. molmol extract and hexane Haddi extract for cytotoxic activity against transformed cells. Extracts were added to medium for each cell line to give a treatment of 1333 ug/ml. Serial dilutions were carried out to enable a range of progressively more dilute concentrations of extract to be tested. The same procedure was followed to make solutions of staurosporine and ethanol of the same concentrations. Staurosporine, an agent with well-established cytotoxic properties, was incorporated into the study to provide a benchmark to compare the cytotoxicity of the extracts against. The ethanol control was carried out because the extracts needed to be dissolved in ethanol prior to introduction into the medium, as the extracts contain mostly organic molecules that would not dissolve in medium alone—this control ensures that it is not the ethanol causing any observed cell death.

100 ul of each concentration of each extract/control were added to 3 separate wells in a labeled microtitre plate. In the untreated control wells 100 ul of medium was added—all wells having a total final volume of 200 ul. 3 identical replicates of this were carried out for testing after 24, 48 and 72 hours. All plates were incubated at 37° C. and 5% CO₂ until reading.

After the required length of time (i.e. 24, 48 or 72 hours) 20 ul MTT solution (5 mg/ml) was added to each well (excluding wells containing only PBS). Plates were then returned to the incubator (under the same conditions) for a further 4 hours. MTT is photosensitive so all plates were covered in foil to minimise exposure to light. After 4 hours all medium from the wells was pipetted out (care was taken to avoid disturbing the formazan crystals) and 200 ul DMSO was added to each well. Careful mixing with a pipette ensured thorough dissolution. All plates were then read on a spectrophotometer for absorption of light at 575 nm. This was done immediately after addition of DMSO. Results are seen in FIGS. 3 to 6.

The method was also employed to test the impact of identified components of the Haddi extracts. Thus, trans-β-Ocimene and γ-Bisabolene both major components of the Haddi extract were tested using the MTT assay to estimate their impact on tumour cell cytotoxicity and viability, see FIGS. 7 to 10.

The cytotoxic effects of ethanol extracted C. molmol and hexane extracted Haddi on S180 fibrosarcoma and HT1080 human fibrosarcoma cell lines are shown in FIGS. 3 to 6. Both C. molmol and Haddi extracts produce a dose dependent increase in cytotoxicity with time. In general the effects of C. molmol extract were found to be more gradual than that of Haddi extract. C. molmol extract shows a gradual increase in cell death after 24hours, whilst Haddi extract results in a more immediate cell death starting at lower doses.

The cytotoxic effects of trans-β-Ocimene and γ-Bisabolene on S180 and HT1080 tumour cell lines is shown in FIGS. 7 to 10.

The cytotoxic effects of trans-β-ocimene, γ-bisabolene and α-bisabolol were also tested on MM6 cells (monocyte leukemia cell line) and A375 cells. The results are shown in Tables 5 to 10 below.

TABLE 1
Determination of Cytotoxicity of trans-β-Ocimene
after 24, 48 and 72 hours on S180 Cells

| | |
|---|---|
| Concentration | % Cytotoxicity |

| (mM) | 24 hours | 48 hours | 72 hours |
|---|---|---|---|
| 250 | 92 ± 0 | 95 ± 0 | 95 ± 0 |
| 200 | 87 ± 3 | 95 ± 1 | 95 ± 0 |
| 150 | 82 ± 7 | 87 ± 5 | 87 ± 5 |
| 100 | 69 ± 11 | 82 ± 7 | 88 ± 4 |
| 50 | 19 ± 3 | 28 ± 4 | 22 ± 5 |

n = 3 replicates of 3

TABLE 2
Determination of Cytotoxicity of trans-β-Ocimene
after 24, 48 and 72 hours on HT1080 Cells

| | |
|---|---|
| Concentration | % Cytotoxicity |

| (mM) | 24 hours | 48 hours | 72 hours |
|---|---|---|---|
| 250 | 67 ± 2 | 90 ± 0 | 90 ± 1 |
| 200 | 62 ± 2 | 90 ± 0 | 90 ± 1 |
| 150 | 43 ± 8 | 71 ± 10 | 64 ± 12 |
| 100 | 16 ± 6 | 33 ± 5 | 15 ± 3 |
| 50 | 1 ± 5 | 0 ± 2 | −5 ± 3 |

* n = 3 replicates of 3

TABLE 3
Determination of Cytotoxicity of γ-Bisabolene
after 24, 48 and 72 hours on S180 Cells

| | |
|---|---|
| Concentration | % Cytotoxicity |

| (mM) | 24 hours | 48 hours | 72 hours |
|---|---|---|---|
| 250 | 89 ± 0 | 89 ± 1 | 86 ± 1 |
| 200 | 90 ± 0 | 93 ± 1 | 89 ± 1 |
| 150 | 92 ± 0 | 93 ± 1 | 91 ± 1 |
| 100 | 93 ± 0 | 95 ± 0 | 94 ± 0 |
| 50 | 93 ± 1 | 95 ± 0 | 94 ± 0 |

* n = 3 replicates of 3

TABLE 4
Determination of Cytotoxicity of γ-Bisabolene
after 24, 48 and 72 hours on HT1080 Cells

| | |
|---|---|
| Concentration | % Cytotoxicity |

| (mM) | 24 hours | 48 hours | 72 hours |
|---|---|---|---|
| 250 | 78 ± 2 | 89 ± 1 | 81 ± 2 |
| 200 | 87 ± 1 | 92 ± 1 | 88 ± 1 |
| 150 | 87 ± 1 | 93 ± 0 | 90 ± 0 |
| 100 | 90 ± 0 | 94 ± 1 | 91 ± 0 |
| 50 | 90 ± 0 | 94 ± 0 | 92 ± 0 |

* n = 3 replicates of 3

It is clear the data that both trans-β-Ocimene and γ-Bisabolene are cytotoxic to S180 fibrosarcoma cells and human tumour cells grown in culture and represent anti-tumour drugs. Ocimene results in almost 100% tumour cell death at concentrations above 200 mM and Bisabolene is potent at concentrations around 50 mM. Both drugs induce apoptosis (see following section). Ocimene is listed as a flavour and is permitted as food additive by the US Food and Drug Administration and is non toxic to mammals.

TABLE 5
Determination of Cytotoxicity of trans-β-ocimene after 24,
48 and 72 hours of MM6 cells (monocyte leukaemic cell line)

| Concentration | % Cytotoxicity | | |
|---|---|---|---|
| (μM) | 24 hours | 48 hours | 72 hours |
| 368 | 57 | 67 | 67 |
| 184 | 46 | 57 | 60 |
| 92 | 32 | 24 | 12 |
| 42 | 15 | −30 | −18 |

TABLE 6
Determination of Cytoxicity of trans-β-ocimene after 24, 48
and 72 hours on A375 cells (a human skin melanoma cell line)

| Concentration | % Cytoxicity | | |
|---|---|---|---|
| (μM) | 24 hours | 48 hours | 72 hours |
| 368 | 29 | 35 | 31 |
| 184 | 7 | 8 | −10 |
| 92 | −11 | −25 | −31 |
| 46 | 7 | −2 | −33 |

TABLE 7
Determination of Cytoxicity of γ-Bisabolene
after 24, 48 and 72 hours on MM6 cells

| Concentration | % Cytoxicity | | |
|---|---|---|---|
| (μM) | 24 hours | 48 hours | 72 hours |
| 10 | 45 | 67 | 78 |
| 2.5 | 51 | 70 | 78 |
| 0.25 | 49 | 68 | 80 |
| 0.13 | 3 | −37 | 13 |

TABLE 8
Determination of Cytoxicity of γ-Bisabolene
after 24, 48 and 72 hours on A375 cells

| Concentration | % Cytoxicity | | |
|---|---|---|---|
| (μM) | 24 hours | 48 hours | 72 hours |
| 10 | 23 | 64 | 77 |
| 2.5 | 15 | 55 | 71 |
| 0.25 | 10 | 7 | 50 |
| 0.13 | 15 | 5 | 13 |

The chemical γ-Bisabolene is a potent anti-tumour agent, inducing high cell death at concentrations as low as 0.25 μM for both tumour cell types.

TABLE 9
Determination of Cytoxicity of α-Bisabolol
after 24 and 72 hours on MM6 cells

| | |
|---|---|
| Concentration | % Cytoxicity |

| (μM) | 24 hours | 72 hours |
|---|---|---|
| 10 | 33 | 49 |
| 2.5 | 46 | 51 |
| 0.25 | 48 | 54 |
| 0.13 | 48 | 53 |

TABLE 10
Determination of Cytoxicity of α-Bisabolol
after 24, 48 and 72 hours on A375 cells

| | |
|---|---|
| Concentration | % Cytoxicity |

| (μM) | 24 hours | 72 hours |
|---|---|---|
| 225 | 31 | 41 |
| 112 | 10 | 26 |
| 56 | −27 | 10 |
| 28 | −2 | 1 |

The chemical α-Bisabolol is a potent anti-tumour agent, inducing high cell death at concentrations as low as 0.13 μM for the MM6 tumour cells. A higher concentration (225 μM) is required to initiate cell death for the A375 tumour cells.

Annexin V Bio assay of apoptosis in HT1080 Fibrosarcoma cells treated with ethanolic C. molmol and hexane Haddi

The Annexin V bioassay is used to distinguish between cells dying via the apoptotic route and cells dying by necrosis. Knowing how ocimene and bisabolene cause cell death is crucial. If the compounds caused necrotic death they would have very little potential as therapeutic agents. This is because necrotic cells burst and release their content into the surroundings, thus causing an inflammatory response that is potentially harmful to neighbouring, non-target cells. Apoptosis on the other hand produces a natural discrete form of cell suicide, which does not cause inflammation.

The Annexin V bioassay utilises an early change in the plasma membrane of apoptotic cells during which phosphatidylserine (PS), a membrane bound phospholipid, is translocated from the inner surface of the membrane bi-layer to the outer surface—thus bringing the PS into contact with the environment. This change occurs before membrane bleeding and far earlier than DNA fragmentation thus making it an ideal marker for early apoptotic cells. Annexin V is a Ca²⁺-dependent phospholipid binding protein with a high affinity for PS. In this study the Annexin V was coupled to a fluorescent marker called FITC, to generate an Annexin V-FITC conjugate. FITC fluoresces with an intense green/yellow colour when viewed via fluorescent microscopy. Thus, early apoptotic cells will bind Annexin V-FITC on their plasma membranes and fluoresce- green/yellow, whereas late apoptotic cells will fluoresce green/yellow in the cytoplasm as well as at the membrane.

To identify necrotic cells another dying agent, propidium iodide (PI) is used. PI is a DNA intercalating agent that stains the chromatin of cells and fluoresces red/orange. As PI is not able to cross an intact plasma membrane it only stains the chromatin of cells that have damaged membranes, i.e. necrotic or late-apoptotic cells that become leaky.

HT1080 cells were grown on cover-slips in 12-well plates at a cell density of 1×10⁶ cells/ml at 37° C. and 5% CO₂. Once established, these cultures were treated with a 0.2% solution of extract (2 μl extract in 2 μl ethanol and 996 μl medium) for 24 hours. An untreated control was also carried out under the same conditions.

Annexin V-FITC and PI staining was carried out according to the manufacturers protocol (Oncogene™ Research Products, Boston USA). All growth medium was removed and cells were washed once with 1 ml PBS and 500 μl binding buffer added to the cells. 10 μl media binding agent (containing CaCl₂ as a source of calcium, as the Annexin V-PS binding is Ca²⁺ dependent) and 1.25 ul Annexin V-FITC was added to the wells. This was then incubated for 15 minutes at room temperature (˜22° C.) in the dark, as Annexin V-FITC is photosensitive. All media was removed by pipetting and 500 μl of cold binding buffer added to the cells. 10 μl of propidium iodide solution was added.

The cover-slip was removed from the well and mounted, cells facing down, on a glass slide using Vectashield™ mounting medium. Cells were then viewed immediately at ×25 magnification on a fluorescent microscope using FITC and rhodamine filters.

### Examples

The Annexin V test below shows the induction of apoptotic cell death in HT1080 human fibrosarcoma cells treated with 5 uM Bisabolene over 12 hours. Cells fluorescing green (shown as white in the Figure) indicate entry into apoptosis, a programmed form of cell death (see FIG. 11).

Similar results were obtained with S180 fibrosarcoma cells and with HT1080 human fibrosarcoma cells treated with 100 uM Ocimene over 12 hours.

The individual chemicals tested to date represent major constituents of scented Myrrh or Haddi. Much work remains to be done on isolating and testing the major constituents of Myrrh or molmol. In this respect particular attention will be given to testing the anti-tumour properties of Furanosesquiterpenoids some of which remain to be identified and characterised.

Normal Fibroblast Cells

Normal fibroblast cells grown in a control culture showed a very low normal occurrence of apoptotic cells, a mean of 3% of a total 400 cells counted, in contrast to 97% normal live cells. When normal fibroblast cells were grown in a medium containing 666 μg/ml ethanolic C. molmol, the production of apoptotic cells was much less than in S180 and HT1080 tumour cell lines, increasing from 4.5% apoptotic cells after 6 hours of incubation to 32% after 24 hours. A similar observation was noted when cells were grown in media containing 234 jig/ml ethanolic C. molmol, though lower percentages of apoptotic cells were observed, increasing from 6.25% after 6 hours of incubation to 19.25% after 24 hours of incubation. See FIG. 12.

In a separate experiment normal fibroblast cells grown in a control culture also showed a slight normal occurrence of apoptotic cells, a mean of 2.25% of a total 400 cells counted, in contrast to 97.75% normal live cells. When normal fibroblast cells were grown in a medium containing 666 μg/ml hexane Haddi extract, the production of apoptotic cells was much less than that obtained in S 180 and HT1080 tumour cell lines, increasing from 16% apoptotic cells after 6 hours of incubation to only 37% after 24 hours. A similar observation was noted when cells grown in media containing 201 μg/ml hexane Haddi extract, though lower percentages of apoptotic cells were observed, increasing from 11% after 6 hours of incubation to only 25.5% after 24 hours of incubation. See FIG. 13.

It is noted that hexane extracted Haddi performed slightly better in producing apoptosis in S180 tumour cells than ethanolic C. molmol. Ethanolic C. molmol extract and hexane Haddi extract treatments produced very similar results in terms of the percentages of apoptotic cells in HT1080 human fibrosarcoma cells. The difference was not significant.

It is clear however that both extracts produce a differentially higher level of apoptosis in the transformed fibrosarcoma cells than in the normal fibrocytes. This means that at suitable doses these extracts are differentially active against tumour cells.

Similar results have been obtained in terms of a differential response to Ocimene and Bisabolene. In addition when mixed cultures containing fibroblast cells and tumour cells are treated, the normal fibroblasts appear to survive while the tumour cells go into apoptosis.

The results of such an experiment are shown in FIG. 14, wherein a mixed tissue culture has been treated for 12 hrs with 5 uM Bisabolene. Note the flattened fibrocytes survive whilst tumour cells round off as apoptotic bodies. In FIG. 15 a mixed tissue culture has been treated for 12 hrs with 100 uM Ocimene. Note fibrocytes spread out and survive whilst tumour cells round off to form apoptotic bodies.

All document cited above are hereby incorporated herein by reference.

1. (canceled)

2. A method of treating or preventing cancer comprising administering to a patient in need of such treatment an effective dose of a compound, wherein the compound is a terpene or oxygenated derivative thereof, and the terpene is selected from the group consisting of:
trans-β-Ocimene; and γ-bisabolene.

3. The method of claim 2, wherein the compound is trans-β-ocimene.

4. The method of claim 2, wherein the compound is γ-bisabolene.

5. The method of claim 2, wherein the compound is administered at a concentration of between 0.2 μM and 100 mM.

6. A pharmaceutical composition comprising the isolated compound: 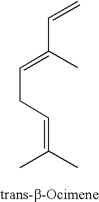
in combination with a pharmaceutically acceptable excipient, carrier, adjuvant or vehicle.

7. The pharmaceutical composition according to claim 6, which is formulated so that a single unit dose of the composition provides trans-β-Ocimene at a concentration of between 0.2 βM and 100 mM.

8. The method of claim 2, wherein the cancer is selected from the group consisting of breast cancer, ovarian cancer, prostate cancer, colon cancer, lung cancer, pancreatic cancer, bowel cancer, melanoma, testicular cancer, cervical cancer, fibrosarcoma, squamous cell carcinoma, leukemia, astrocytoma and glioma.

9. A method of making a medicament for the treatment or prophylaxis of cancer comprising: combining a Commiphora guidotti extract with a pharmaceutically acceptable excipient, carrier, adjuvant, or vehicle.

10. (canceled)

11. The method of claim 2, wherein the compound is an isolated compound.
